(19) United States
(12) Patent Application Publication
(10) Pub. No.: US 2025/0146963 A1
(43) Pub. Date: May 8, 2025

(54) PREPARATION METHOD OF TEST PIECE FOR FLAME-RESISTANCE EVALUATION, AND TEST PIECE FOR FLAME-RESISTANCE EVALUATION

(71) Applicant: Sumitomo Riko Company Limited, Aichi (JP)

(72) Inventors: Shigehiro Otsubo, Aichi (JP); Satoru Ogawa, Aichi (JP)

(73) Assignee: Sumitomo Riko Company Limited, Aichi (JP)

(21) Appl. No.: 19/014,227

(22) Filed: Jan. 9, 2025

Related U.S. Application Data
(63) Continuation of application No. PCT/JP2022/036283, filed on Sep. 28, 2022.

(51) Int. Cl. G01N 25/20

(52) U.S. Cl. CPC G01N 25/20

(57) ABSTRACT

A preparation method of a test piece 100 with respect to an axle beam bush formed of metal and rubber with different material properties includes: setting a predetermined region S, so that a surface area of a portion exposed to a heat source at a predetermined position and having a lowest flame resistance is maximized in an actual use state of the axle beam bush; calculating the surface area ratio of each member in the region; and preparing the test piece 100 by using a metal member 103 and a rubber member 104 as materials same as respective members in the region, so that the metal member 103 and the rubber member 104 are exposed at ratios same as the calculated surface area ratios.

### CROSS-REFERENCE TO RELATED APPLICATION

The present application is a continuation of PCT/JP2022/036283, filed on Sep. 28, 2022. The entire contents of the aforementioned application is hereby incorporated by reference herein.

### BACKGROUND

### Technical Field

The disclosure relates to a preparation method of a test piece for flame-resistance evaluation suitable for an evaluation test based on the European standard of EN45545-2 for flame resistance and a test piece for flame-resistance evaluation prepared by using the preparation method.

### Description of Related Art

The European standard EN45545-2 for flame resistance is a unified European standard set for the purpose of unifying the fire protection standards among the respective countries, and is required for all parts related to railway vehicles. Such standard is not limited to Europe, but adopted across the world. In Japan as well, evaluations in accordance with such standard are carried out.

For example, in Patent Document 1, a method in which a sheet-shaped test piece is cut out from a molded article formed of a resin composition used in an interior material for railway vehicles, etc., and the maximum average heat generation rate is evaluated in accordance with EN45545-2 is disclosed.

In Patent Document 2, a method for evaluating heat generation properties and smoke generation properties of synthetic leather used for vehicle seats in accordance with EN45545-2 is disclosed.

In Patent Document 3, a method in which a flexible member used in an air spring for railway vehicles is cut in a predetermined shape, and a heat generation test defined in EN45545-2 is carried out on the obtained test piece to evaluate flame resistance.

### PRIOR ART DOCUMENT(S)

### Patent Document(s)

Patent Document 1: Japanese Laid-open No. 2019-38895
Patent Document 2: Japanese Laid-open No. 2020-12252
Patent Document 3: Japanese Patent No. 7014562

Clause 5.3.2 of EN45545-2 sets forth the following: “The test must be performed under an exposed surface condition same as the actual use condition.”

If the evaluation target is a molded article formed of the same member or a sheet or a member formed of the same material no matter which part is cut out, like the test pieces disclosed in Patent Documents 1 to 3, the flame-resistance evaluation performed with the test piece can be performed under the exposed surface condition same as the actual use conditions.

However, in the case where the evaluation target is a composite member formed by combining members of different materials (e.g., metal and rubber), such as a bush or an axle spring used in a railway truck, it is not possible to cut out a test piece locally, making it difficult to properly evaluate the composite member as a whole.

Therefore, the disclosure provides a preparation method of a test piece for flame resistance capable of properly evaluating the composite member as a whole by assuming actual use even when the evaluation target is a composite member formed by combining members of different materials. A test piece for flame resistance is also provided.

### SUMMARY

An aspect of the disclosure provides: a preparation method of a test piece for flame-resistance evaluation with respect to a composite member formed of multiple members of different materials. The preparation method includes: setting a predetermined region, so that in an actual use state of the composite member, a surface area of a portion exposed to a heat source at a predetermined position and having a lowest flame resistance is maximized; calculating a surface area ratio of each of the members in the predetermined region; and preparing the test piece by using multiple test members formed of materials same as those of the respective members in the predetermined region, so that the test members are exposed at ratios same as the surface area ratios that are calculated.

### BRIEF DESCRIPTION OF THE DRAWINGS

FIG. 1 is a schematic view illustrating an actual use state of an axle beam bush of Embodiment 1.

FIGS. 2A to 2C are schematic views illustrating a test piece for flame-resistance evaluation of Embodiment 1, where FIG. 2A is a plan view, FIG. 2B is a cross-sectional view taken along a line A-A, and FIG. 2C is a cross-sectional view taken along a line A-A of a modified example.

FIG. 3 is a schematic view illustrating an actual use state of a single link bush of Embodiment 2.

FIGS. 4A to 4D are schematic views illustrating a test piece for flame-resistance evaluation of Embodiment 2, where FIG. 4A is a plan view, FIG. 4B is a cross-sectional view taken along a line B-B, FIG. 4C is a cross-sectional view taken along a line B-B of a modified example, and FIG. 4D is a plan view of a modified example.

FIG. 5 is a schematic view illustrating an actual use state of an axle spring of Embodiment 3.

FIGS. 6A to 6D are schematic views illustrating a test piece for flame-resistance evaluation of Embodiment 3, where FIG. 6A is a plan view, FIG. 6B is a cross-sectional view taken along a line C-C, FIG. 6C is a cross-sectional view taken along a line C-C of a modified example, and FIG. 6D is a plan view of a modified example.

FIG. 7 is a schematic view illustrating an actual use state of an axle spring of Embodiment 4.

FIGS. 8A to 8B are schematic views illustrating a test piece for flame-resistance evaluation of Embodiment 4, where FIG. 8A is a plan view, and FIG. 8B is a cross-sectional view taken along a line D-D.

FIGS. 9A to 9B are schematic views illustrating a modified example of the test piece for flame-resistance evaluation of Embodiment 4, where FIG. 9A is a plan view, and FIG. 9B is a cross-sectional view taken along a line E-E.

FIGS. 10A to 10B are schematic views illustrating a modified example of the test piece for flame-resistance evaluation in which a coating layer is formed on a front surface of a sample layer, where FIG. 9A is a plan view, and FIG. 9B is a cross-sectional view taken along a line F-F.

FIGS. 11A to 11B are schematic views illustrating a modified example of a test piece for flame-resistance evaluation illustrating another arrangement example of a metal member and a rubber member, where FIG. 11A is a plan view, and FIG. 11B is a cross-sectional view taken along a line G-G.

### DESCRIPTION OF THE EMBODIMENTS

A first configuration of the disclosure relates to a preparation method of a test piece for flame-resistance evaluation with respect to a composite member formed of multiple members of different materials. The preparation method includes: setting a predetermined region, so that in an actual use state of the composite member, a surface area of a portion exposed to a heat source at a predetermined position and having a lowest flame resistance is maximized; calculating the surface area ratio of each member in the predetermined region; and preparing the test piece by using multiple test members formed of materials same as those of the respective members in the predetermined region, so that the test members are exposed at ratios same as the surface area ratios that are calculated.

According to another aspect of the first configuration, in the above configuration, an arrangement of the respective test members in the test piece is same as an arrangement of the respective members in the predetermined region.

According to another aspect of the first configuration, in the above configuration, the test piece is arranged in a predetermined shape with a front surface being a flat surface, one of the test members is arranged on an outer side of the flat surface in a surface direction, and an other of the test members is arranged on an inner side of the front surface in the surface direction.

According to another aspect of the first configuration, in the above configuration, a single surface of the test piece is prepared to expose at the same ratios by using the respective test members.

According to another aspect of the first configuration, in the above configuration, both surfaces of the test piece are respectively prepared to expose at the same ratios by using the respective test members.

According to another aspect of the first configuration, in the above configuration, in a case where the composite member has a cavity on an inner side of a front surface, a cavity is also formed on an inner side of a front surface of the test piece.

According to another aspect of the first configuration, in the above configuration, in a case where the composite member is formed of metal and rubber, the test piece is prepared by setting the predetermined region to maximize a surface area of the rubber.

In order to achieve the above objective, a second configuration of the disclosure relates to a test piece for flame-resistance evaluation with respect to a composite member formed of multiple members of different materials. The test piece is prepared by using the preparation method.

According to the disclosure even if the evaluation target is a composite member formed by combining members of different materials, the composite member can be properly evaluated as a whole by using the test piece for flame-resistance evaluation that reproduces the assumed actual use.

According to another aspect of the disclosure, in addition to the above effects, the arrangement of the respective test members in the test piece is the same as the arrangement of the respective members in the predetermined region. Therefore, a test piece close to the actual use state of the composite member can be prepared.

According to another aspect of the disclosure, in addition to the above effects, the test piece is arranged in a predetermined shape with a front surface being a flat surface, one of the test members is arranged on an outer side of the flat surface in a surface direction, and an other of the test members is arranged on an inner side of the front surface in the surface direction. Therefore, a test piece close to the actual use state of the composite member can be prepared.

According to another aspect of the disclosure, in addition to the above effects, a single surface of the test piece is prepared to expose at the same ratios by using the respective test members. Therefore, a test piece that reproduces the actual use state can be easily prepared.

According to another aspect of the disclosure, in addition to the above effects, both surfaces of the test piece are respectively prepared to expose at the same ratios by using the respective test members. Therefore, a test piece can be set without caring the front-back orientation at the time of performing the evaluation test, and a mistake at the time of setting can be eliminated.

According to another aspect of the disclosure, in addition to the above effects, in a case where the composite member has a cavity on an inner side of a front surface, a cavity is also formed on an inner side of a front surface of the test piece. Therefore, a test piece close to the actual use state of the composite member having a cavity can be prepared.

According to another aspect of the disclosure, in addition to the above effects, in a case where the composite member is formed of metal and rubber, the test piece is prepared by setting the predetermined region to maximize a surface area of the rubber. Therefore, the test piece that reproduces the actual use state in the least favorable situation can be obtained.

In the following, the embodiments of the disclosure will be described based on the drawings.

### Embodiment 1

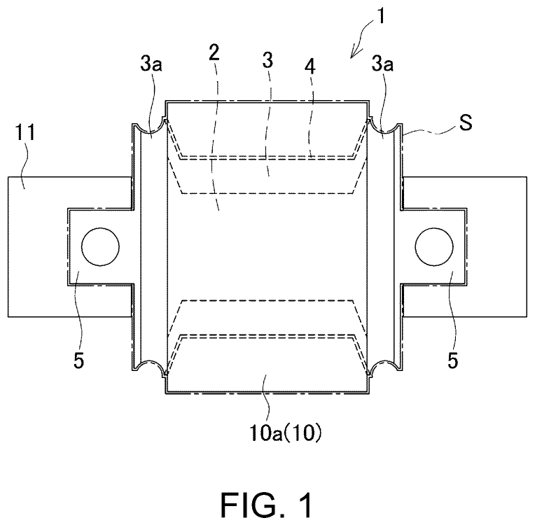

FIG. 1 illustrates an axle beam bush 1 used in an axle beam truck as a railway vehicle. The axle beam truck has a structure in which an upper-lower load is supported by a spring on an axle box, and a front-rear load is supported by an axle beam connecting the axle box and a truck frame. The axle beam bush 1 is incorporated into a connection part between an axle beam 10 and a truck frame 11, and serves to transmit the load while also exerts a vibration isolation function.

FIG. 1 illustrates in a bottom view the actual use state of the axle beam bush 1 as the evaluation target. The axle beam bush 1 includes an axle-shaped inner metal fitting 2 located at the center, a rubber 3 bonded to the outer peripheral surface of the inner metal fitting 2, and an outer metal fitting 4 bonded to the outer peripheral surface of the rubber 3. Boss parts 5, 5 provided on two axial end parts of the inner metal fitting 2 are supported by the truck frame 11, and an intermediate portion of the axle beam bush 1 including the outer metal fitting 4 is supported by a cylindrical part 10a provided at an end part of the axle beam 10.

Accordingly, when it is assumed that the heat source is on the ground (near the rail), the respective lower half peripheral portions of the metallic cylindrical part 10a, end parts 3a, 3a of the rubber 3 protruding from the cylindrical part 10a, and the boss parts 5, 5 of the inner metal fitting 2 formed of metal are exposed to the heat source.

Taking into consideration the actual use state of the axle beam bush 1, a method for preparing a test piece for evaluating flame-resistance (simply referred to as “test piece” in the following) will be described.

Firstly, a region is set so that the surface area of the rubber 3 exposed to the heat source at the lower part and having the lowest flame resistance is maximized. In such case, a region S surrounded by a dot-chain line is set.

Then, the surface area ratios of the exposed portions of the cylindrical part 10a of the axle beam 10 formed of metal, the left and right boss parts 5, 5 that are metallic, and the end parts 3a, 3a of the rubber 3 in the region S are respectively calculated. For example, the ratios are as follows.

### [Surface Area Ratios]

Axle beam (cylindrical part): 25000 mm² (47%)
Boss part: 20000 mm² (38%)
Rubber: 8000 mm² (15%)
===
Total: 53000 mm²

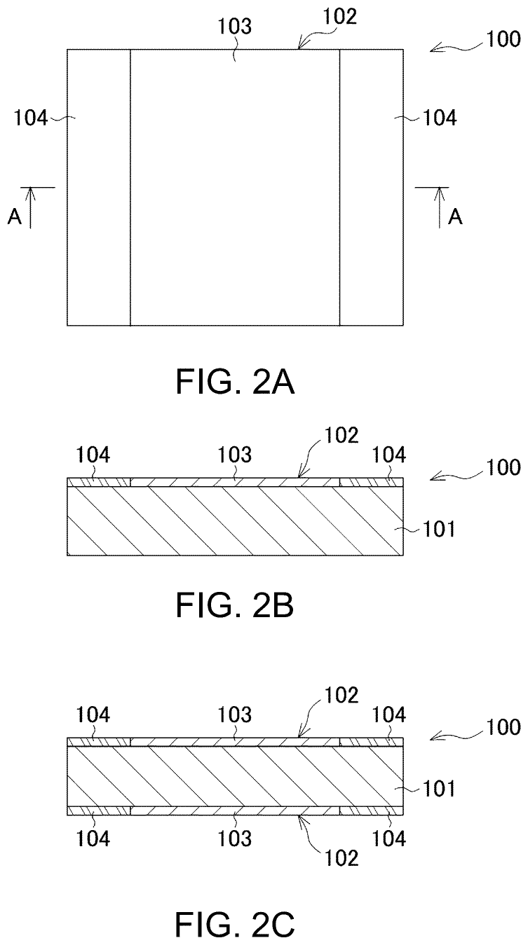

Then, a test piece defined in EN45545-2 (e.g., a test piece in a size of 75 mm (length)×75 mm (width)×25 mm (thickness)) is prepared. At this time, as shown in FIGS. 2A and 2B, the test piece 10, on the front surface of a main body rubber 101 serving as a lower layer, has a sample layer 102 formed by using two test members, i.e., metal (e.g., iron) and rubber (e.g., natural rubber) same as the materials of the respective members in the region S. The main body rubber 101 may be formed of the same material as or a different material from the sample layer 102. In the sample layer 102, metal and rubber are exposed at the ratios same as the calculated surface area ratios in the region S. The arrangement of the two members also matches the actual use state.

Accordingly, in the sample layer 102, a metal member 103 is formed at the center at a ratio of 85% or less, and rubber members 104 on the left and right of the metal member 103 together are formed at a ratio of 15% or more. In this way, the test piece 100 having on the front surface the sample layer 102 reproducing the actual use state can be obtained.

Nevertheless, as shown in FIG. 2C, the sample layer 102 may also be formed on the front and back surfaces, instead of being formed on only a single surface. In this way, if both sides of the test piece 100 are respectively prepared to expose at the same ratios by using the metal member 103 and the rubber member 104, the test piece 100 can be set without caring the front-back orientation when the evaluation test is performed, and a mistake at the time of setting can be eliminated.

It is noted that, in EN45545-2, the thickness of the metal portion of the test piece is defined. For example, a steel plate is 0.8 mm in thickness, and an aluminum plate is 1.0 mm. Accordingly, in the sample layer 102 as well, the thickness of the metal portion is set based on EN45545-2.

In this way, if a test is performed by using the test piece 100 in which the sample layer 102 is formed on the front surface, the flame-resistance evaluation can be performed by using the exposed surface condition same as the actual use condition.

In this way, as a preparation method of the test piece 100 for the axle beam bush 1 (an example of the composite member) formed of metal and rubber (an example of multiple members) of different materials in Embodiment 1, the procedures are as follows: the predetermined region S is set, so that, in the actual use state of the axle beam bush 1, the surface area of the portion exposed to the heat source at a predetermined position and having the lowest flame resistance is maximized; the surface area ratio of each member in the region S is calculated; and the test piece 100 is prepared by using the metal member 103 and the rubber member 104 (an example of multiple test members) as materials same as the materials of the respective members in the region S, so that the metal member 103 and the rubber member 104 are exposed at ratios same as the calculated surface area ratios.

According to the configuration, even if the axle beam bush 1 as the evaluation target is a composite member formed by combining members of different materials, the composite member can be properly evaluated as a whole by using the test piece 100 that reproduces the assumed actual use.

In particular, the arrangement of the metal member 103 and the rubber member 104 in the test piece 100 is the same as the arrangement of the respective members in the region S. Accordingly, the test piece 100 close to the actual use state of the axle beam bush 1 can be prepared.

A single surface of the test piece 100 is prepared so that the metal member 103 and the rubber member 104 are exposed by using the same ratios.

Accordingly, the sample layer 102 that reproduces the actual use state can be easily prepared.

In the axle beam bush 1 formed by metal and rubber, the test piece 100 is prepared by setting the region S so that the surface area of rubber is maximized.

Accordingly, the test piece 100 that reproduces the actual use state in the least favorable situation can be obtained.

### Embodiment 2

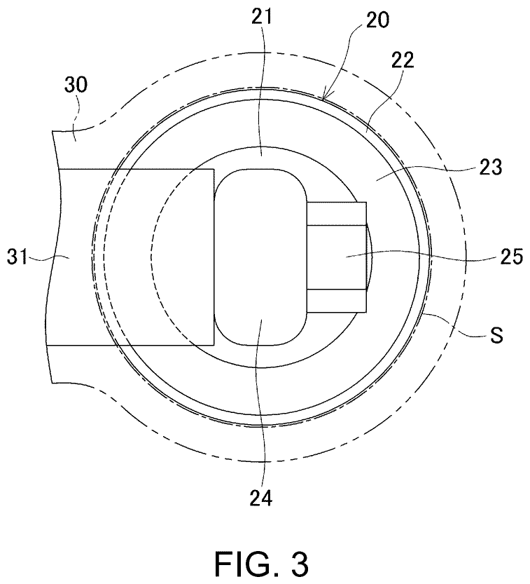

FIG. 3 illustrates a single link bush 20 used in a traction device for railway vehicles. The single link bush 20 is incorporated into the connection parts at the two ends of a single link (monolink) 30 connecting a truck and a vehicle body to transmit the load and exert a vibration isolation function.

FIG. 3 illustrates the actual use state of the single link brush 20 as the evaluation target from a lateral side. The single link bush 20 is incorporated into an end part of the single link, and includes an inner metal fitting 21 having an axle shape and located at the center, an outer metal fitting 22 having a cylindrical shape and located outer of the inner metal fitting 21, and a rubber 23 having a cylindrical shape and bonded to the two metal fittings 21, 22 between the inner metal fitting 21 and the outer metal fitting 22. A connection part 24 protruding laterally from the end part of the inner metal fitting 21 is connected with a truck frame 31 by using a bolt 25.

Accordingly, when the heat source is assumed to be located on a lateral side (on the front side in the direction intersecting with the paper surface of FIG. 3) with respect to the connection part 24, the truck frame 31 formed of metal, the connection part 24, the bolt 25, and the inner metal fitting 21, the rubber 23 on the outer side thereof, and the outer metal fitting 22 are respectively exposed to the heat source.

Taking into consideration the actual use state of the single link bush 20, a method for preparing a test piece will be described.

Firstly, the region S (a region surrounded by a dot-chain line) is set so that the surface area of the rubber 23 exposed to the heat source and having the lowest flame resistance is maximized.

Then, the surface area ratios of the exposed portions of the truck frame 31 formed of metal, the connection part 24, the bolt 25, the inner metal fitting 21, the rubber 23 (the C-shaped portion not overlapped with the truck frame 31), and the outer metal fitting 22 in the region S are respectively calculated. For example, the ratios are as follows. The truck frame 31, the connection part 24, the bolt 25, the inner metal fitting 21, and the outer metal fitting 22 are generally calculated as the metal part.

### [Surface Area Ratios]

Metal part: 20000 mm² (66.6%)
Rubber: 10000 mm² (33.3%)
===
Total: 30000 mm²

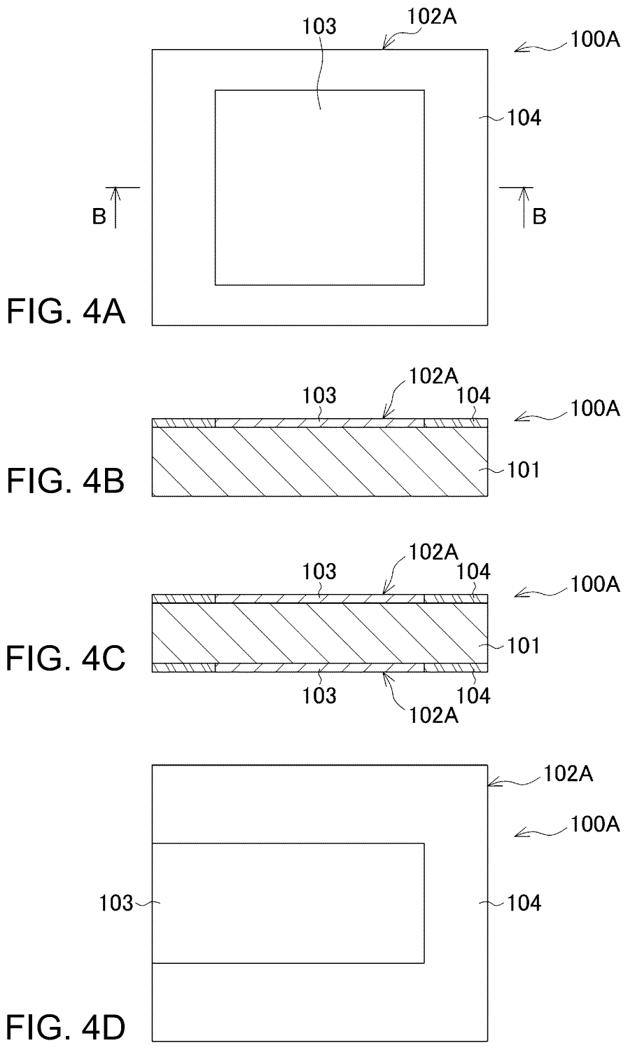

Then, a test piece defined in EN45545-2 (e.g., a test piece in a size of 75 mm (length)×75 mm (width)×25 mm (thickness)) is prepared. At this time, as shown in FIGS. 4A and 4B, a test piece 100A, on the front surface of the main body rubber 101 serving as a lower layer, has a sample layer 102A formed by using two test members, i.e., metal (e.g., iron) and rubber (e.g., natural rubber) same as the materials of the respective members in the region S. In the sample layer 102A, metal and rubber are exposed at the ratios same as the calculated surface area ratios in the region S. The thickness of the metal portion is set based on EN45545-2.

In the sample layer 102A, the metal member 103 is formed at the center, which serves as the inner side in the surface direction, at a ratio of 66.6% or less, and the rubber member 104 is formed on the outer periphery of the metal member 103, which serves as the outer side in the surface direction, at a ratio of 33.3% or more. In this way, the test piece 100A having on the front surface the sample layer 102A that reproduces the actual use state can be obtained. However, as shown in FIG. 4C, the sample layer 102A may also be formed on the front and back surfaces, instead of being formed only on the front surface. Moreover, considering that the rubber 23 is exposed in a C-shape, in the sample layer 102A, the metal member 103 may also extend to be long on a single side to cut out a lateral side of the rubber member 104, as shown in FIG. 4D.

In this way, if a test is performed by using the test piece 100A in which the sample layer 102A is reproduced on the front surface, the flame-resistance evaluation can be performed by using the exposed surface condition same as the actual use condition.

In this way, as a preparation method of the test piece 100A for the single link bush 20 (an example of the composite member) formed of metal and rubber (an example of multiple members) of different materials in Embodiment 2 as well, the procedures are as follows: the predetermined region S is set, so that, in the actual use state of the single link bush 20, the surface area of the portion exposed to the heat source at the predetermined position and having the lowest flame resistance is maximized; the surface area ratio of each member in the region S is calculated; and the test piece 100A is prepared by using the metal member 103 and the rubber member 104 (an example of multiple test members) as materials same as the materials of the respective members in the region S, so that the metal member 103 and the rubber member 104 are exposed at ratios same as the calculated surface area ratios.

According to the configuration, even if the single link bush 20 as the evaluation target is a composite member formed by combining members of different materials, the composite member can be properly evaluated as a whole by using the test piece 100A that reproduces the assumed actual use, and the same effect like Embodiment 1 can be achieved.

In particular, the test piece 100A is arranged so that, as the predetermined shape in which the front surface is a flat surface, the rubber member 104 (an example of a test member) is arranged on the outer side of the flat surface in the surface direction and the metal member 103 (an example of an other test member) on the inner side of the front surface in the surface direction.

Accordingly, the test piece 100A close to the actual use state of the single link bush 20 can be prepared.

### Embodiment 3

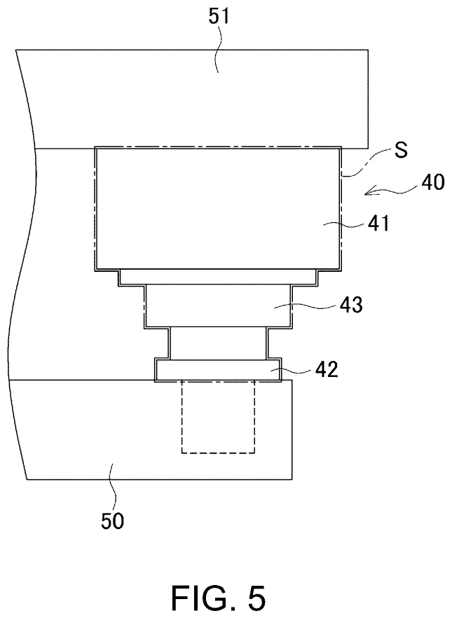

FIG. 5 illustrates an axle spring 40 used in a connection part between an axle box and a truck frame of railway vehicles. The axle spring 40 elastically supports a truck frame 51 on an axle box 50, and serves to transmit a load from the truck frame 51 toward the axle box 50 while also exerts a vibration isolation function.

FIG. 5 illustrates the actual use state of the axle spring 40 as the evaluation target from a lateral view. The axle spring 40 includes an upper metal fitting 41 having a cylindrical shape and fixed to the truck frame 51, a lower metal fitting 42 fixed to the axle box 50, and a laminated rubber 43 having a cone shape and connecting the upper metal fitting 41 and the lower metal fitting 42. Accordingly, when the heat source is assumed to be at the front or lateral side, the entirety or a half of the periphery of the upper metal fitting 41 and the lower metal fitting 42 formed of metal and the laminated rubber 43 is respectively exposed to the heat source.

Taking into consideration the actual use state of the axle spring 40, a method for preparing a test piece will be described.

Firstly, the region S (a region surrounded by a dot-chain line) is set, so that the surface area of the rubber 43 exposed to the heat source and having the lowest flame resistance is maximized.

Then, the surface area ratios of the exposed portions of the upper metal fitting 41 and the lower metal fitting 42 formed of metal and the laminated rubber 43 in the region S are respectively calculated. For example, the ratios are as follows. Here, the respective ratios are calculated by using the entire periphery. The upper metal fitting 41 and the lower metal fitting 42 are generally calculated as the metal part.

### [Surface Area Ratios]

Metal part: 100000 mm² (41%)
Rubber: 140000 mm² (59%)
===
Total: 240000 mm²

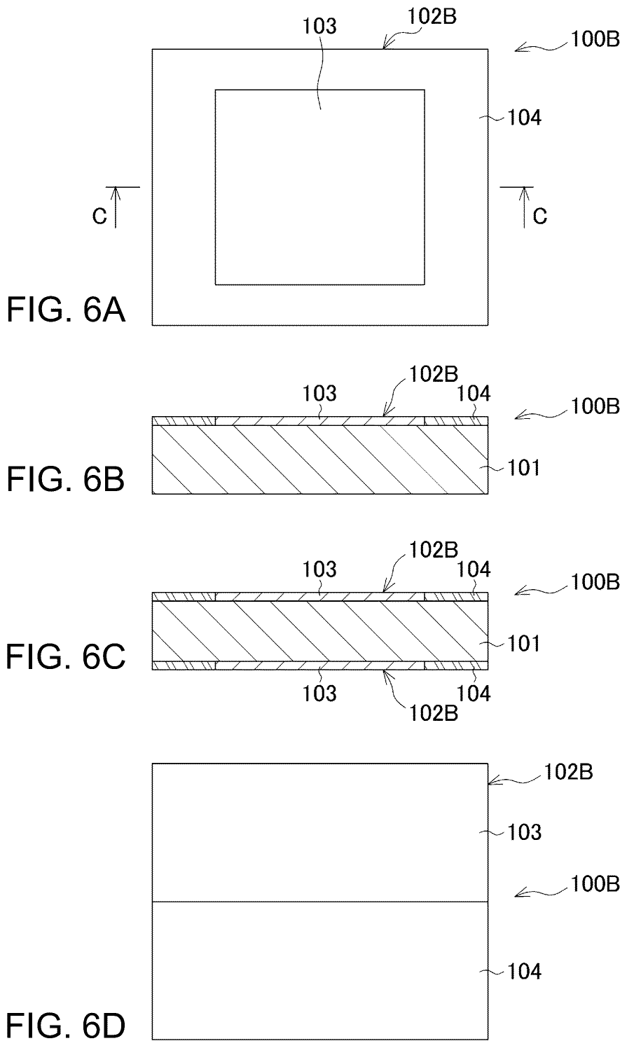

Then, a test piece defined in EN45545-2 (e.g., a test piece in a size of 75 mm (length)×75 mm (width)×25 mm (thickness)) is prepared. At this time, as shown in FIGS. 6A and 6B, a test piece 100B, on the front surface of the main body rubber 101 serving as a lower layer, has a sample layer 102B formed by using two test members, i.e., metal (e.g., iron) and rubber (e.g., natural rubber), same as the materials of the respective members in the region S. In the sample layer 102B, metal and rubber are exposed at the ratios same as the calculated surface area ratios in the region S. The thickness of the metal portion is set based on EN45545-2.

In the sample layer 102B, the metal member 103 is formed at the center, which serves as the inner side in the surface direction, at a ratio of 41% or less, and the rubber member 104 is formed on the outer periphery of the metal member 103, which serves as the outer side in the surface direction, at a ratio of 59% or more. In this way, the test piece 100B having the sample layer 102B that reproduces the actual use state on the front surface can be obtained. However, as shown in FIG. 6C, the sample layer 102B may also be formed on the front and back surfaces, instead of being formed only on the front surface. Also, the sample layer 102B is not limited to the configuration in which the metal member 103 is surrounded by the rubber member 104. It may also be that a half is arranged as the metal member 103 and a half is arranged as the rubber member 104, as shown in FIG. 6D.

In this way, if a test is performed by using the test piece 100B in which the sample layer 102B is reproduced on the front surface, the flame-resistance evaluation can be performed by using the exposed surface condition same as the actual use condition.

In this way, as a preparation method of the test piece 100B for the axle spring 40 (an example of the composite member) formed of metal and rubber (an example of multiple members) of different materials in Embodiment 3 as well, the procedures are as follows: the predetermined region S is set, so that, in the actual use state of the axle spring 40, the surface area of the portion exposed to the heat source at a predetermined position and having the lowest flame resistance is maximized; the surface area ratio of each member in the region S is calculated; and the test piece 100B is prepared by using the metal member 103 and the rubber member 104 (an example of multiple test members) as materials same as the materials of the respective members in the region S, so that the metal member 103 and the rubber member 104 are exposed at ratios same as the calculated surface area ratios.

According to the configuration, even if the axle spring 40 as the evaluation target is a composite member formed by combining members of different materials, the composite member can be properly evaluated as a whole by using the test piece 100B that reproduces the assumed actual use, and the same effect like Embodiments 1 and 2 can be achieved.

### Embodiment 4

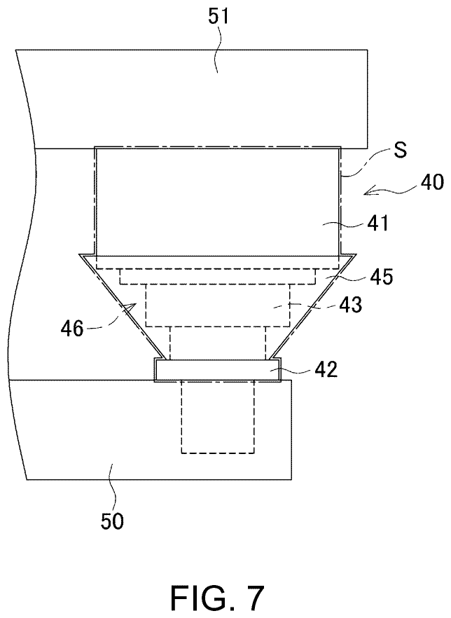

Although FIG. 7 illustrates the axle spring 40 same as the axle spring of Embodiment 3, the embodiment differs from Embodiment 3 in the point that a cover 45 formed of fire-resistant rubber and covering the entire laminated rubber 43 is installed. A cavity (gap) 46 having a cylindrical shape throughout the entire periphery is formed between the cover 45 and the laminated rubber 43.

Accordingly, when the heat source is assumed to be at the front or lateral side, the entirety or a half of the periphery of the upper metal fitting 41 and the lower metal fitting 42 formed of metal and the cover 45 is exposed to the heat source.

Taking into consideration the actual use state of the axle spring 40, a method for preparing a test piece will be described.

Firstly, the region S (a region surrounded by a dot-chain line) is set, so that the surface area of the cover 45 exposed to the heat source and having the lowest flame resistance is maximized.

Then, the surface area ratios of the exposed portions of the upper metal fitting 41 and the lower metal fitting 42 formed of metal and the cover (rubber) 45 in the region S are respectively calculated. For example, the ratios are as follows. Here, the respective ratios are calculated by using the entire periphery. The upper metal fitting 41 and the lower metal fitting 42 are generally calculated as the metal part.

### [Surface Area Ratios]

Metal part: 100000 mm² (50%)
Rubber: 100000 mm² (50%)
===
Total: 200000 mm²

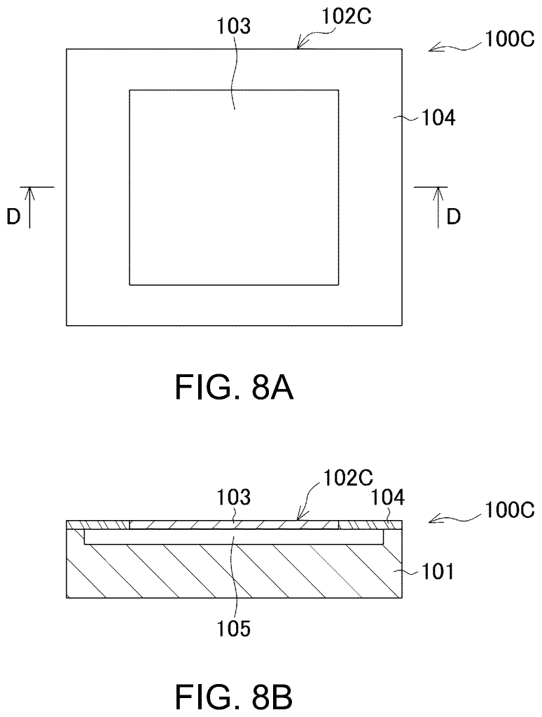

Then, a test piece defined in EN45545-2 (e.g., a test piece in a size of 75 mm (length)×75 mm (width)×25 mm (thickness)) is prepared. At this time, as shown in FIGS. 8A and 8B, a test piece 100C, on the front surface of the main body rubber 101 serving as a lower layer, has a sample layer 102C formed by using two test members, i.e., metal (e.g., iron) and rubber (e.g., CSM rubber) same as the materials of the respective members in the region S. In the sample layer 102C, metal and rubber are exposed at the ratios same as the calculated surface area ratios in the region S. The thickness of the metal portion is set based on EN45545-2.

In the sample layer 102C, the metal member 103 is formed at the center, which serves as the inner side in the surface direction, at a ratio of 50% or less, and the rubber member 104 is formed on the outer periphery of the metal member 103, which serves as the outer side in the surface direction, at a ratio of 50% or more. In addition, considering that the cavity 46 is formed between the laminated rubber 43 and the cover 45, as shown in FIG. 8B, a cavity (e.g., in a thickness of 6 mm) 105 is formed between the metal member 103 as well as the rubber member 104 and the main body rubber 101. The size (area and depth) of the cavity 105 can be set based on, for example, the ratio with respect to the total volume of the laminated rubber 43, the average of the distance between the cover 45 and the laminated rubber 43, the dimension between the cover 45 and the laminated rubber 43 at a portion that is most likely to be affected by the heat source, etc., by considering the extent of the cavity 46 in the actual axle spring 40.

In the case of FIGS. 8A, 8B as well, the sample layer 102C and the cavity 105 may also be formed on the front and back surfaces.

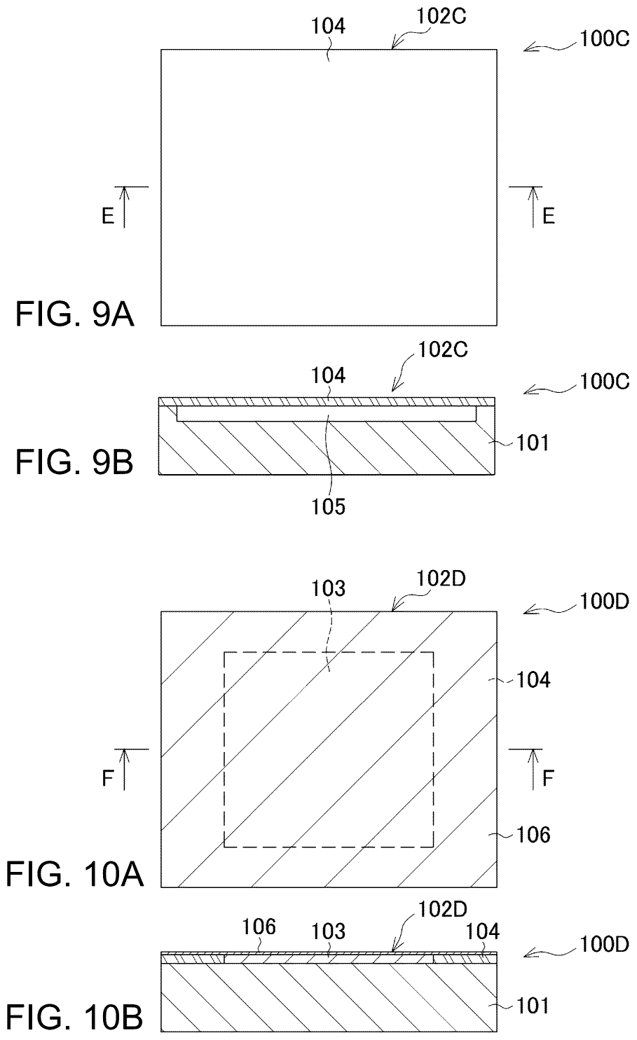

However, in order to perform evaluation as an unfavorable condition, as shown in FIGS. 9A and 9B, in the sample layer 102C of the test piece 100C, the metal member can be omitted to only provide the rubber member 104.

In this way, if a test is performed by using the test piece 100C in which the sample layer 102C is reproduced on the front surface, the flame-resistance evaluation can be performed by using the exposed surface condition same as the actual use condition.

In this way, as a preparation method of the test piece 100C for the axle spring 40 (an example of the composite member) formed of metal and rubber (an example of multiple members) of different materials in Embodiment 4 as well, the procedures are as follows: the predetermined region S is set, so that, in the actual use state of the axle spring 40, the surface area of the portion exposed to the heat source at a predetermined position and having the lowest flame resistance is maximized; the surface area ratio of each member in the region S is calculated; and the test piece 100C is prepared by using the metal member 103 and the rubber member 104 (an example of multiple test members) as materials same as the materials of the respective members in the region S, so that the metal member 103 and the rubber member 104 are exposed at ratios same as the calculated surface area ratios.

According to the configuration, even if the axle spring 40 as the evaluation target is a composite member formed by combining members of different materials, the composite member can be properly evaluated as a whole by using the test piece 100C that reproduces the assumed actual use, and the same effect like Embodiments 1 to 3 can be achieved.

In particular, since the axle spring 40 has the cavity 46 on the inner side of the front surface, the cavity 105 is formed also on the inner side of the front surface of the test piece 100C. Accordingly, the test piece 100C close to the actual use state of the axle spring 40 having the cavity 46 can be prepared.

In the following, examples of modifications shared among the respective embodiments will be described.

In the case where a coating layer is formed on the front surface of the evaluation target by applying a flame-resistant paint, as shown in FIGS. 10A and 10B, for example, in a test piece 100D in which a sample layer 102D formed of the metal member 103 and the rubber member 104 is prepared as well, a coating layer 106 can be formed on the front surface of the sample layer 102. However, the configuration of the sample layer 102D itself is not limited to FIGS. 10A and 10HB. The configuration of the arrangement of the metal member and the rubber member can be modified as appropriate in accordance with the structure of the evaluation target. The shape of the member disposed at the center of the sample layer in a plan view, like FIG. 4A, is not limited to being square or rectangular, and may also be other shapes, such as a circle. The arrangement of the metal member and the rubber member may be reversed from that in the above embodiment.

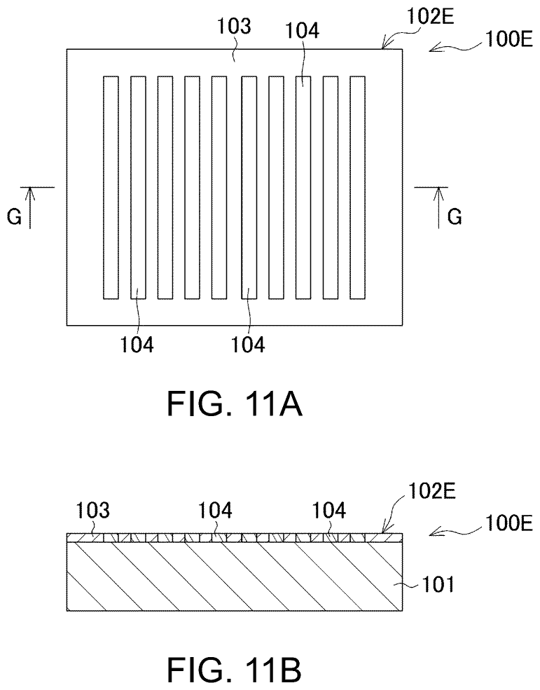

As shown in a test piece 100E in FIGS. 11A and 11B, it may also be arranged that multiple band-shaped rubber members 104 are disposed in parallel on the inner side of the metal member 103. The shape and the size of the test piece can be changed as appropriate, as long as the test piece can be used in an evaluation test based on EN45545-2. For example, a test piece in which the shape is circular in a plan view, a test piece in which the thickness of the main body rubber is reduced, or a test piece with only the sample layer may be prepared.

In the case where an unevenness or stepped difference is present on the exposed surface of the evaluation target, the unevenness or stepped difference may also be reproduced in the sample layer.

If a recess exceeds the thickness of the test piece, a through hole can also be formed in the test piece.

The members forming the composite member are not limited to two members. It is possible to prepare a test piece according to the disclosure even if there are three or more members. That is, it suffices as long as the test piece is prepared by using a test member fitting the number of the composite member.

What is claimed is:

1. A preparation method of a test piece for flame-resistance evaluation with respect to a composite member formed of a plurality of members of different materials, the preparation method comprising:
setting a predetermined region, so that in an actual use state of the composite member, a surface area of a portion exposed to a heat source at a predetermined position and having a lowest flame resistance is maximized;
calculating a surface area ratio of each of the members in the predetermined region; and
preparing the test piece by using a plurality of test members formed of materials same as those of the respective members in the predetermined region, so that the test members are exposed at ratios same as the surface area ratios that are calculated.

2. The preparation method as claimed in claim 1, wherein an arrangement of the respective test members in the test piece is same as an arrangement of the respective members in the predetermined region.

3. The preparation method as claimed in claim 1, wherein the test piece is arranged in a predetermined shape with a front surface being a flat surface, one of the test members is arranged on an outer side of the flat surface in a surface direction, and an other of the test members is arranged on an inner side of the front surface in the surface direction.

4. The preparation method as claimed in claim 1, wherein a single surface of the test piece is prepared to expose at the same ratios by using the respective test members.

5. The preparation method as claimed in claim 1, wherein both surfaces of the test piece are respectively prepared to expose at the same ratios by using the respective test members.

6. The preparation method as claimed in claim 1, wherein, in a case where the composite member has a cavity on an inner side of a front surface, a cavity is also formed on an inner side of a front surface of the test piece.

7. The preparation method as claimed in claim 1, wherein, in a case where the composite member is formed of metal and rubber, the test piece is prepared by setting the predetermined region to maximize a surface area of the rubber.

8. A test piece for flame-resistance evaluation with respect to a composite member formed of a plurality of members of different materials,
wherein the test piece is prepared by using the preparation method as claimed in claim 1.
